(19) United States
(12) Patent Application Publication
(10) Pub. No.: US 2026/0328879 A1
(43) Pub. Date: Oct. 22, 2026

(54) Preparation of a T2-Weighted Magnetization in an Examination Object for Magnetic Resonance Imaging

(71) Applicant: Siemens Healthineers AG, Forchheim (DE)

(72) Inventor: Alto Stemmer, Uttenreuth (DE)

(73) Assignee: Siemens Healthineers AG, Forchheim (DE)

(21) Appl. No.: 19/468,069

(22) Filed: Feb. 3, 2026

(30) Foreign Application Priority Data
Feb. 3, 2025 (DE) 10 2025 103 825.2

(51) Int. Cl. G01R 33/50; G01R 33/36; G01R 33/56; G01R 33/561

(52) U.S. Cl. CPC G01R 33/50; G01R 33/3607; G01R 33/5617; G01R 33/5608

(57) ABSTRACT

The disclosure is directed to a method for preparation of a T2-weighted magnetization in an examination object for magnetic resonance imaging. The method may include irradiating an RF excitation pulse for generating a transversal magnetization in the examination object, after irradiation of the RF excitation pulse, irradiating at least two RF refocusing pulses for refocusing the generated magnetization, so after each RF refocusing pulse, a spin echo is formed, and a duration, characteristic of the prepared T2-weighting, after the irradiation of the RF excitation pulse and after the most recent irradiated RF refocusing pulse, irradiating an RF pulse for returning the generated transversal magnetization into the longitudinal. The at least two RF refocusing pulses are irradiated in such a way that an echo time of a first refocused spin echo is shorter than the characteristic duration divided by the number of irradiated RF refocusing pulses.

### CROSS REFERENCE TO RELATED APPLICATIONS

This patent application claims priority to German Patent Application No. 102025103825.2, filed Feb. 3, 2025, which is incorporated herein by reference in its entirety.

### BACKGROUND

The disclosure relates to an improved preparation of a T2-weighted magnetization for magnetic resonance imaging.

The magnetic resonance technique (hereinafter the abbreviation MR stands for magnetic resonance) is a known technique with which images of the inside of an examination object can be generated. Put simply, for this, the examination object is positioned in a magnetic resonance device in a comparatively strong static, homogeneous main magnetic field, also called B0 field, with field strengths of 0.2 tesla to 7 tesla and more, so the nuclear spins thereof are oriented along the main magnetic field. For triggering nuclear spin resonances which can be measured as signals, radio-frequency pulses (RF pulses) are irradiated into the examination object in order to manipulate the nuclear spins, the triggered nuclear spin resonances are measured as what is known as k-space data by means of coils designed to receive and on the basis of this, MR images are reconstructed or spectroscopy data is ascertained. The magnetic alternating field generated by the excitation pulses irradiated by means of at least one transmit coil is also referred to as a B1 field. For spatial encoding of the measurement data, fast-switched magnetic gradient fields, called gradients for short, are overlaid on the main magnetic field. A pattern that is used, which describes a temporal sequence of RF pulses to be irradiated and gradients to be switched, is referred to as a pulse sequence (pattern), or also as a sequence for short. The recorded measurement data is digitized and stored in a k-space matrix as complex numerical values. An associated MR image can be reconstructed from the k-space matrix furnished with values, for example by means of a multidimensional Fourier transform.

RF pulses, which are played out by the pulse sequences, have to be amplified to powers of the order of magnitude of kilowatts. This is the task of a radio-frequency power amplifier (RFPA). A maximum power demand of the RFPA is scaled with the Larmor frequency squared. That is to say, in order to achieve a specific B1 field, a higher power is required at a magnetic resonance device with a higher field strength of the main magnetic field. If, therefore, RF pulses of a magnetic resonance device are scaled with a lower field strength of the main magnetic field, for example of 0.55 T (Larmor frequency~23 MHz) for a magnetic resonance device with a higher field strength of the main magnetic field, for example with 1.5 T (Larmor frequency~63 MHz), then RF pulses or RF pulse sequences previously used by the pulse sequences may possibly exceed the power of an RFPA of the magnetic resonance device with the higher field strength and it is thus no longer possible to achieve the RF pulses or pulse sequences on the magnetic resonance device with the higher field strength.

The following correlation between a tilt angle α, about which nuclear spins manipulated by the RF pulse are tilted, and a B1 field of the RF pulse exists for conventional RF pulses:

$$α = γ {\int }_{0}^{T} {B}_{1} ( t ) d t \qquad ( 1 )$$

In this case, γ is the gyromagnetic ratio and the integral extends over the period T of the RF pulse.

It is often difficult to generate a constant B1 field over the entire image volume. According to formula 1, the tilt angle varies thereby. If the desired tilt angle is not achieved, then this generally has an adverse effect on the image quality.

Adiabatic RF pulses are a special class of RF pulses which can uniformly excite, refocus or invert magnetization, even if the B1 field of the pulses varies spatially. Therefore the correlation, described by formula 1, between tilt angle and B1 field does not apply to adiabatic RF pulses. Instead, the tilt angle of an adiabatic RF pulse depends on how the amplitude and frequency (or phase) of the B1 field is varied over time during the irradiation of the RF pulse. In this case, the modulation over time of amplitude and frequency differs according to whether an RF pulse is to be used as an excitation pulse, inversion pulse or refocusing pulse. As a rule, adiabatic RF pulses are much longer (8-30 ms) and have a higher B1 amplitude than conventional RF pulses and thereby place particularly high demands on RFPAs.

T2-FLAIR (“Fluid Attenuated Inversion Recovery”) is a standard technique in neuroimaging which is often used for examinations of the head. With T2-FLAIR, cerebrospinal fluid (CSF) is suppressed with an inversion pulse (IR) in order to prevent the bright CSF signal from concealing lesions. The repetition times TR for T2-FLAIR techniques lie in the order of magnitude of 10 seconds in order to achieve a desired contrast between white and gray matter. 2D-TSE sequences (TSE: “Turbo Spin Echo”) are typically used in the T2-FLAIR technique. The 2D techniques are effective since, due to intelligent nesting techniques, the majority of the TR time can be used for imaging. Unfortunately however, the 2D techniques show artifacts as a consequence of unsuppressed CSF which flows into the measured slice between inversion and excitation. The artifacts can be prevented by 3D techniques and the use of non-selective inversion pulses. However, owing to the long repetition time TR, 3D T2-FLAIR techniques are deemed to be inefficient. The combination of a plurality of technical developments makes 3D T2-FLAIR possible in a clinically acceptable time: apart from specific 3D TSE sequences, which with the aid of short non-selective RF refocusing pulses reduce the echo spacing, and with specific tilt angle evolutions lengthen the usable duration of a TSE echo train which is used and parallel imaging techniques (ppa techniques), such as 3D CAIPIRINHA (“Controlled Aliasing in Parallel Imaging Results in Higher Acceleration”), which reduce the number of the k-space data points, which have to be sampled for the image reconstruction, immediately before an imaging module, i.e., for example, immediately before an inversion pulse of a FLAIR sequence, it is possible to use a T2 preparation module which allows the repetition time TR to be shortened.

Apart from sequences with inversion pulse, such as a FLAIR sequence, other sequences, in particular imaging sequences, can also benefit from use of a T2 preparation module immediately before their execution. Thus, for example, the contrast can be manipulated by a T2-preparation module, as is described, for example, in the article by Mugler et al., “Improved T2-weighted Imaging of the pelvis using T2-prepared Single-slab 3D TSE (SPACE)”, Proc. Intl. Soc. Mag. Reson. Med. 19; p. 833, 2011, and/or undesirable background signals can be reduced, as is described, for example, in the article by Busse et al., “Improved Background Suppression for 3D-MRCP using T2-Prep”, Proc. Intl. Soc. Mag. Reson. Med. 14: p. 392, 2006.

T2-preparation modules of this kind for the preparation of a T2-weighted magnetization for a subsequent imaging sequence and the power demand thereof on RFPAs are the subject of the following statements.

As a rule, a T2-preparation module consists of an 90° RF excitation pulse, a variable number of RF refocusing pulses and a final 90° RF flip-back pulse, also called “tip-up pulse”.

The excitation pulse generates a transversal magnetization, which is singly or multiply refocused with the aid of the refocusing pulse(s), and is turned back or flipped-back into the longitudinal by the final 90° RF pulse. A spoiler gradient can be used after the final 90° pulse in order to dephase a remaining transversal magnetization. Since the magnetization between the two 90° pulses, i.e. the 90° RF excitation pulse and the final 90° RF flip-back pulse, is subject to the T2-decay, the longitudinal magnetization is T2-weighted immediately after the T2-preparation module. The T2-contrast of an imaging sequence executed immediately thereafter is, for example, amplified thereby.

The characteristic time of the T2-preparation module is the time between the centers of the two 90° pulses, while that of the magnetization is subject to the T2-decay. This time is called the T2-preparation duration and abbreviated below to T_{T2P}.

If a T2-preparation module is used in connection with a FLAIR technique, the T2-preparation module is thus executed immediately before the inversion pulse of the FLAIR sequence. CSF has a long T2-time and is accordingly barely attenuated by the T2-preparation modules. Gray and white matter, by contrast, have T2-times which are shorter than the T2-preparation duration, and are consequently almost saturated immediately after the T2-preparation module. Roughly speaking, after the inversion pulse, CSF follows an inversion recovery while gray and white matter are subject to a faster saturation recovery. This reduces an undesirable T1-weighting of images reconstructed from MR measurement data recorded in this way. More details on this are described, for example, in the article by Visser et al., “High-Resolution Magnetization-Prepared 3D-FLAIR Imaging at 7.0 Tesla”, Magn. Reson. Med. 64: p. 194-202, 2010, which describes for the first time the use of a T2-preparation module with a FLAIR technique. The T2-preparation module described there consists of one non-selective 90° RF rectangular pulse each for excitation and flipping-back of the magnetization. A phase of the 90° RF flip-back pulse is shifted by 180° compared to the phase of the 90° RF excitation pulse. Four identical non-selective hyperbolic secant pulses are used as refocusing pulses. Hyperbolic secant pulses are the adiabatic pulses most often used for an inversion. The time interval between the centers of the six pulses τ″−2τ″−2τ″−2τ″−τ″ respectively, for τ″=T_{T2P}/8, that is to say τ″ equal to the time interval between the initial 90° RF excitation pulse and the first RF hyperbolic secant pulse, or τ/2−τ−τ−τ−τ/2, for τ=T_{T2P}/4, and thus τ equal to the time interval between two hyperbolic secant pulses.

A further T2-preparation module, which, instead of four hyperbolic secant pulses, uses four adiabatic RF refocusing pulses of the BIREF-1 type (B1-insensitive adiabatic refocusing pulse), is described, for example, in the article by Jenista et al. “Motion and Flow Insensitive Adiabatic T2-Preparation Module for Cardiac MR Imaging at 3 Tesla”, Magn. Reason. Med. 70: p. 1360-1368, 2013.

### BRIEF DESCRIPTION OF THE DRAWINGS/FIGURES

The accompanying drawings, which are incorporated herein and form a part of the specification, illustrate the embodiments of the present disclosure and, together with the description, further serve to explain the principles of the embodiments and to enable a person skilled in the pertinent art to make and use the embodiments.

FIG. 1 shows a schematic graph to illustrate an example of the time correlations of the RF pulses to be irradiated of a T2-preparation module with four adiabatic RF pulses for refocusing.

FIG. 2 shows a schematic graph to illustrate an example of the time correlations of the RF pulses to be irradiated of a T2-preparation module with two adiabatic RF pulses for refocusing.

FIG. 3 is a flowchart of a method for the preparation of a T2-weighted magnetization in an examination object according to an exemplary embodiment of the disclosure.

FIG. 4 shows a schematic graph to illustrate the time correlations of the RF pulses to be irradiated of an inventive T2-preparation module with four RF pulses for refocusing according to an exemplary embodiment of the disclosure.

FIG. 5 shows a schematic graph to illustrate the time correlations of the RF pulses to be irradiated of an inventive T2-preparation module with two RF pulses for refocusing according to an exemplary embodiment of the disclosure.

FIG. 6 shows a magnetic resonance system according to an exemplary embodiment of the disclosure.

The exemplary embodiments of the present disclosure will be described with reference to the accompanying drawings. Elements, features and components that are identical, functionally identical and have the same effect are—insofar as is not stated otherwise—respectively provided with the same reference character.

### DETAILED DESCRIPTION

In the following description, numerous specific details are set forth in order to provide a thorough understanding of the embodiments of the present disclosure. However, it will be apparent to those skilled in the art that the embodiments, including structures, systems, and methods, may be practiced without these specific details. The description and representation herein are the common means used by those experienced or skilled in the art to most effectively convey the substance of their work to others skilled in the art. In other instances, well-known methods, procedures, components, and circuitry have not been described in detail to avoid unnecessarily obscuring embodiments of the disclosure. The connections shown in the figures between functional units or other elements can also be implemented as indirect connections, wherein a connection can be wireless or wired. Functional units can be implemented as hardware, software or a combination of hardware and software.

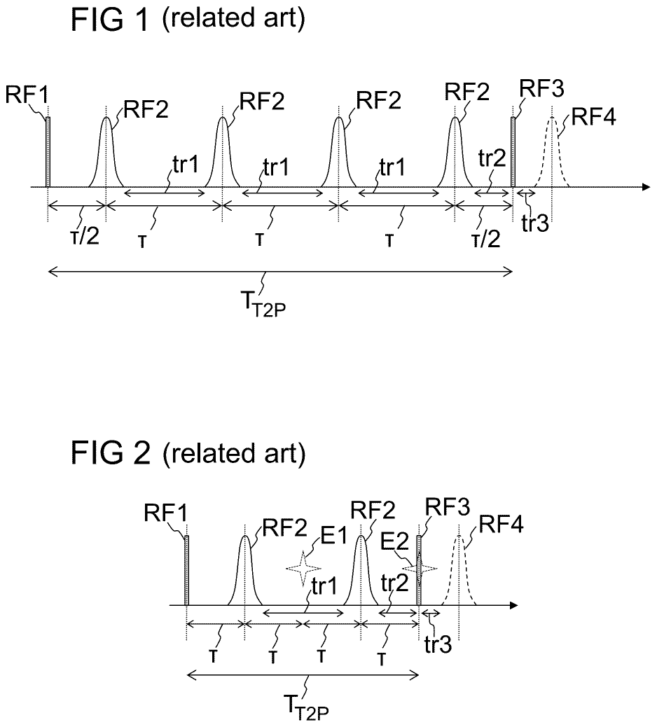

FIG. 1 shows a schematic graph to illustrate the time correlations of the RF pulses to be irradiated of a T2-preparation module with four adiabatic RF pulses for refocusing. After a period τ/2 following irradiation of a 90° RF excitation pulse RF1, four RF refocusing pulses RF2 respectively are irradiated in the time interval of τ, followed by a final 90° RF flip-back pulse RF3 after a duration of t/2 after the most recent RF refocusing pulse RF2, so the characteristic duration of the preparation module T_{T2P} can be written as: T_{T2P}=4τ. A first RF pulse RF4 of an imaging sequence following the preparation module is also represented in FIG. 1. The first RF pulse RF4 of the following imaging sequence can be, in particular, an RF inversion pulse, for example a FLAIR sequence. As a rule, an RF inversion pulse of this kind is also an adiabatic RF pulse which places a similar demand on the charging account of the RFPA as a single RF refocusing pulse RF2 of the T2-preparation module. The first RF pulse RF4 of the imaging sequence can also be a further RF excitation pulse, for example a TSE sequence. The first RF pulse RF4 of the imaging sequence is irradiated immediately, that is to say as soon as possible, after the T2-preparation module.

A further T2-preparation module with two adiabatic RF refocusing pulses in the time interval of TE/2 and a time interval TE/4 between the 90° RF excitation pulse and the first RF refocusing pulse, as well as between the second RF refocusing pulse and the 90° RF flip-back pulse, whereby the echo time TE here is equal to the previously defined T2-preparation duration T_{T2P}, is described in U.S. Pat. No. 7,787,930 B2.

FIG. 2 shows a schematic graph to illustrate the time correlations of the RF pulses to be irradiated of a T2-preparation module with two adiabatic RF pulses for refocusing. A first primary spin echo E1 is formed after a period τ=T_{T2P}/4 following the first RF refocusing pulse RF2. The interval between this primary spin echo E1 and the second RF refocusing pulse RF2 is again τ=T_{T2P}/4, so at the instant T_{T2P}/4 after the second RF refocusing pulse RF, a second spin echo E2 is formed. At this instant the 90° RF flip-back pulse is switched in order to reverse the magnetization back into the transversal plane.

The echo time of the primary spin echo E1 here is therefore TE1=2τ=2T_{T2P}/4=T_{T2P}/2.

The echo time of the second spin echo E2 here is TE2=4 τ=TE1+2T_{T2P}/4=T_{T2P}/2+T_{T2P}/2=T_{T2P}.

In other words, after a period τ following irradiation of a 90° RF excitation pulse RF1, two RF refocusing pulses RF2 are irradiated in the time interval of 2τ, followed by a final 90° RF flip-back pulse RF3 after a duration of τ following the most recent RF refocusing pulse RF2, so the characteristic duration of the preparation module T_{T2P} can again be written as: T_{T2P}=4τ. A first RF pulse RF4 of an imaging sequence following the preparation module is also represented in FIG. 2. The first RF pulse RF4 of the following imaging sequence can again be, in particular, an RF inversion pulse, for example a FLAIR sequence. As a rule, an RF inversion pulse of this kind is also an adiabatic RF pulse, which places a similar demand on the charging account of the RFPA as a single RF refocusing pulse RF2 of the T2-preparation module. The first RF pulse RF4 of the imaging sequence is irradiated immediately, that is to say as soon as possible, after the T2-preparation module.

The RFPA of a magnetic resonance device is generally not designed in such a way that the power required during transmission of an RF pulse can be called up on a sustained basis. Instead, use is made of the fact that the required average power is significantly lower. The energy for the RF pulses is delivered by a capacitor bank. The capacitor bank is continuously recharged during transmission of RF pulses and in (transmission) breaks between the RF pulses. The charging balance of an RFPA may be modelled accordingly by what is known as a charging balance model, as is described, for example, in U.S. Pat. No. 10,145,919 B2.

The RFPA is described by a charging account q(T) from which each RF pulse is “deducted”. As long as the charging account is positive, the maximum power is available at the output of the RFPA. The removal of charge Δq of a pulse is proportional to the magnitude integral (voltage integral) of the RF pulse. The proportionality factor c takes into account a reflection-contingent “derating” of the RFPA and further details of the transmission chain. An RF pulse shall be described here by its N support points uᵢ in the time interval Δt. The voltage integral is therefore:

$${\sum }_{i = 1}^{N} {u}_{i} Δ t$$

The change in the charging account Δq due to an RF pulse of total duration N×Δt in the model is thereby:

$$Δ q = - c {\sum }_{i = 1}^{N} {u}_{i} Δ t + κ N Δ t \qquad ( 1 )$$

In this case, κ is the recharging rate of the RFPA/charging account and proportional to the ratio of average and peak output power of the RFPA:

$$κ ∼ \frac{{P}_{a v g}}{{P}_{p e a k}} .$$

Recharging takes place at the same rate K between the RF pulses until a maximum charge Q₀ is reached.

The following consequences are produced thereby for a design of a pulse sequence:
1. An RF pulse for which Δq≥0 can be repeated as often as desired.
2. An RF pulse for which Δq<−Q₀ cannot be executed even with a fully charged capacitor bank.
3. For all other RF pulses (where −Q₀≤Δq<0), the following applies: whether the RF pulse can be transmitted or not depends on the status of the charging account immediately before the start of the RF pulse.

An attained T2-weighting of the longitudinal magnetization after a 90° RF flip-back pulse of a T2-preparation module is dependent on the T2-preparation duration T_{T2P}, i.e. the time between initial 90° RF excitation pulse and 90° RF flip-back pulse.

If the condition 3 (−Q₀≤Δq<0) for an RF refocusing pulse applies, the demand on the RFPA can be lowered by a reduction in the number of RF refocusing pulses because the recharging time between two RF refocusing pulses of the T2-preparation module and, with an inversion pulse following the T2-preparation module, such as with a subsequent FLAIR sequence, the recharging time between a most recent RF refocusing pulse of the T2-preparation module and the (adiabatic) inversion pulse following the T2-preparation module increases thereby.

As may be seen from FIGS. 1 and 2, in the case of a T2-preparation module with a duration of T_{T2P} with four RF refocusing pulses RF2 according to the article by Jenista et al. mentioned above, the recharging time between two RF refocusing pulses is, for example,

$$t r 1 = \frac{{T}_{T 2 P}}{4} - {T}_{refoc} ,$$

where T_{refoc} is the duration of an RF refocusing pulse RF2. In the case of a T2-preparation module of the same duration T_{T2P}, for example according to U.S. Pat. No. 7,787,930 B2, which has already been mentioned, with two RF refocusing pulses RF2, the recharging time is extended to

$$\frac{{T}_{T 2 P}}{2} - {T}_{refoc} .$$

A recharging time between a most recent RF refocusing pulse RF2 of a T2-preparation module and a possibly following inversion pulse RF4 of

$$tr 2 = \frac{{T}_{T 2 P}}{4} - \frac{{T}_{refocP}}{2}$$

for the variant with four RF refocusing pulses RF2 would accordingly increase to

$$tr 2 = \frac{{T}_{T 2 P}}{8} - \frac{{T}_{refoc}}{2}$$

for the variant with two RF refocusing pulses RF2 by

$$\frac{{T}_{T 2 P}}{4} - \frac{{T}_{T 2 P}}{8} = \frac{{T}_{T 2 P}}{8} .$$

In both cases, a time interval tr3 between the final 90° RF flip-back pulse RF3 and the first RF pulse RF4 of a subsequent imaging sequence is, as a rule, determined by a duration of a spoiler gradient (not represented) switched in a known manner in this period in order to prevent undesirable signal paths, and for typical T2-preparation durations T_{T2P} is short compared to the respective times tr1 and tr2.

If non-selective rectangular pulses are used for excitation by the 90° RF excitation pulse and for flipping-back by the 90° RF flip-back pulse, a load of a RFPA charging account due to non-selective rectangular pulses of this kind is low compared to a load due to adiabatic RF pulses, in particular adiabatic RF refocusing pulses, and is usually ignored in the following considerations.

In order to minimize the load of an RFPA, a design of a T2-preparation module with a single RF refocusing pulse would be obvious. However, this limits the class of the adiabatic RF pulses which can be used. A distinction is made between adiabatic RF inversion pulses and adiabatic RF refocusing pulses. The task of an adiabatic RF inversion pulse is to tilt the longitudinal magnetization from the +z-axis (i.e. aligned with the static B₀ field) into the −z axis (i.e. directed counter to the B₀ field). An adiabatic RF refocusing pulse rotates a transversal (or longitudinal) magnetization by 180° about an axis in the transversal plane. An adiabatic RF refocusing pulse is characterized in that it rotates all spins irrespective of their phasing in the transversal plane by 180° (i.e. even when the magnetization has already been dephased since the excitation), so at a later instant, an echo is formed. If, by contrast, an adiabatic RF inversion pulse is used for refocusing, the magnetization dephases during the pulse. The consequence of this is an extra phase at the instant of the echo (i.e. at the instant of the 90° RF flip-back pulse), which results in the magnetization, also those isochromates whose T2-time is long compared with the T2-preparation duration, being only incompletely flipped-back into the transversal plane. If, by contrast, two identical adiabatic RF inversion pulses are used for refocusing, then the second RF inversion pulse completely compensates the phase dispersion generated by the first RF inversion pulse.

The hyperbolic secant pulse belongs to the class of adiabatic RF inversion pulses. If, therefore, there is a desire to use hyperbolic secant pulses in a T2-preparation module, the number of hyperbolic secant pulses used for refocusing has to be even.

An object of the disclosure is to enable improved preparation of a T2-weighted magnetization, which, in particular, reduces the load of a charging account of an RFPA without increasing a B1-sensitivity of the T2-preparation, so, in particular, adiabatic RF pulses can be used in the preparation of the T2-weighting of the magnetization.

The object is achieved by a method for preparation of a T2-weighted magnetization in an examination object for magnetic resonance imaging, a magnetic resonance system, a computer program, and an electronically readable data carrier.

An inventive method for preparation of a T2-weighted magnetization in an examination object for magnetic resonance imaging may comprise the following steps:
irradiation of an RF excitation pulse for generating a transversal magnetization in the examination object,
after irradiation of the RF excitation pulse, irradiation of at least two RF refocusing pulses for refocusing the generated magnetization, so after each RF refocusing pulse, a spin echo is formed,
a duration, characteristic of the prepared T2-weighting, after the irradiation of the RF excitation pulse and after the most recent irradiated RF refocusing pulse, irradiation of an RF pulse for returning the generated transversal magnetization into the longitudinal, wherein the at least two RF refocusing pulses are irradiated in such a way that an echo time of a first refocused spin echo is shorter than the characteristic duration divided by the number of irradiated RF refocusing pulses.

The inventive shortening of the echo time of the first spin echo extends a recharging time tr2 between the most recent irradiated RF refocusing pulse RF2 and the RF pulse RF3 for returning the magnetization. The thus longer possible recharging of a capacitor bank up to the RF pulse RF3 for returning the magnetization means it can be charged further and thus has more charge available for subsequent RF pulses.

An inventive magnetic resonance system may comprise a magnet unit, a gradient unit, a radio-frequency unit and a control facility (controller), configured to carrying out an inventive method, with a preparation unit.

Am inventive computer program implements an inventive method on a control facility when it is executed on the control facility. By way of example, the computer program comprises commands which, when the program is executed by a control facility, for example a control facility of a magnetic resonance system, prompt this control facility to execute an inventive method. The control facility can be designed in the form of a computer.

The computer program can also be in the form of a computer program product which can be loaded directly into a memory of a control facility, with program code means in order to execute an inventive method when the computer program product is executed in a computing unit of a computing system of the control facility.

An inventive computer-readable storage medium comprises commands which, when executed by a control facility, for example a control facility of a magnetic resonance system, prompt it to execute an inventive method.

The computer-readable storage medium can be embodied as an electronically readable data carrier which comprises electronically readable items of control information stored thereon, which comprise at least one inventive computer program and are embodied in such a way that they carry out an inventive method when the data carrier is used in a control facility of a magnetic resonance system.

The advantages and statements disclosed in relation to the method also apply analogously to the magnetic resonance system, the computer program product and the electronically readable data carrier.

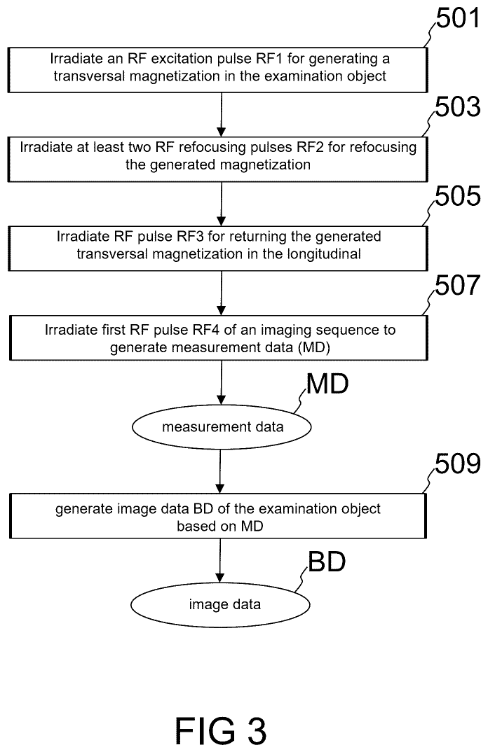

FIG. 3 shows a schematic flowchart of an inventive method for the preparation of a T2-weighted magnetization in an examination object U for magnetic resonance imaging.

An RF excitation pulse RF1 for generating a transversal magnetization in the examination object U is irradiated into the examination object U (Block 501).

After the irradiation of the RF excitation pulse RF1, at least two RF refocusing pulses RF2 for refocusing the generated magnetization are irradiated, so after each RF refocusing pulse RF2, a spin echo E1, E2, E3 or E4 is formed (Block 503).

A duration T_{T2P}, characteristic of the prepared T2-weighting, after the irradiation of the RF excitation pulse RF1 and after the most recent irradiated RF refocusing pulse RF2, an RF pulse RF3 for returning the generated transversal magnetization in the longitudinal is irradiated into the examination object (Block 505).

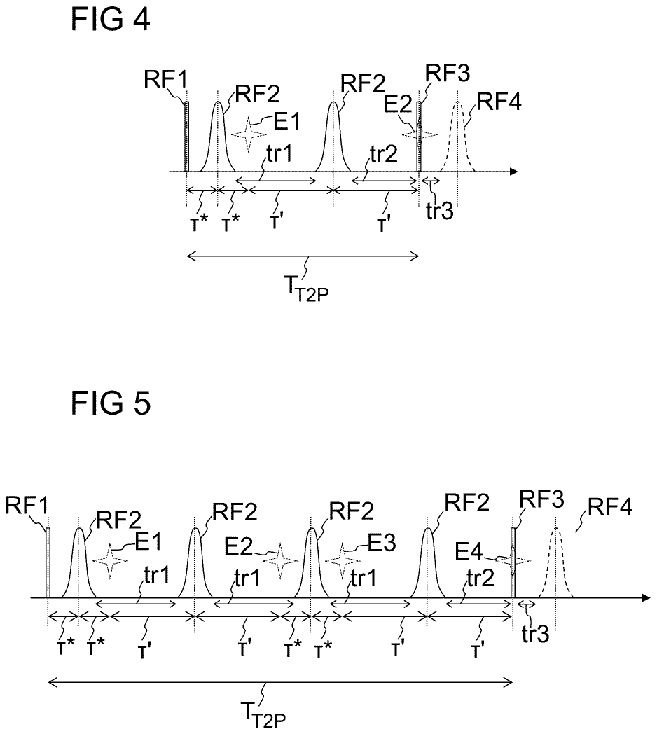

The at least two RF refocusing pulses RF2 are irradiated in such a way that an echo time of a first refocused spin echo E1 is shorter than the characteristic duration T_{T2P} divided by the number of irradiated RF refocusing pulses RF2. More details on this will be explained below with reference to FIGS. 4 and 5.

Immediately after the RF pulse RF3 for returning the generated transversal magnetization in the longitudinal, a first RF pulse RF4 of an imaging sequence can be irradiated. Immediately after the preparation of the T2-weighted magnetization, the imaging sequence can thus be begun and carried out for capturing, with measurement data MD being recorded (Block 507), on the basis of which image data BD of the examination object can be generated (Block 509).

FIG. 4 shows a schematic graph to illustrate the time correlations of the RF pulses to be irradiated of an inventive T2-preparation module with two adiabatic RF pulses for refocusing. Compared to the graph in FIG. 2, the first RF refocusing pulse RF2 here is irradiated as early as after a period τ*, where τ*<τ, so an echo time TE1 of the first spin echo E1 is double this period τ*, TE1=2τ*. The echo time TE1 of the first spin echo E1 here is thus also shorter than the echo time (TE1=2T=T_{T2P}/2) of the first spin echo E1 according to FIG. 2. Here the echo time TE1 of the first refocused spin echo E1 is thus shorter than the characteristic duration T_{T2P} divided by the number (here: two) of the irradiated RF refocusing pulses RF2.

The primary spin echo E1 is, as in the example of FIG. 2, refocused by the second RF refocusing pulse RF2, so at the instant T_{T2P}−TE1 after the primary spin echo E1, a second spin echo E2 is formed. The echo time TE2 of the second spin

$$TE 2 = TE 1 + 2 \times ( \frac{{T}_{T 2 P}}{2} - \frac{TE 1}{2} ) = {T}_{T 2 P}$$

is unchanged compared to conventional techniques. In addition, compared to the known example in FIG. 2, both RF refocusing pulses RF2 can be shifted, for example by

$$\frac{{T}_{T 2 P}}{4} - \frac{TE 1}{2} ,$$

forwards in time. The time interval between the two RF refocusing pulses RF2, and therewith the recharging time of the RFPA between the two RF refocusing pulses RF2, is unchanged and equal to

$$tr 1 = \frac{{T}_{T 2 P}}{2} - {T}_{refoc} .$$

In this case, T_{refoc} is again the duration of an RF refocusing pulse RF2.

The shorter recharging time tr2 between the second RF refocusing pulse RF2 and the 90° RF flip-back pulse is, by contrast, extended in FIG. 4 compared to the example in FIG. 2 and, more precisely, by

$$Δ tr 2 = \frac{{T}_{T 2 P}}{2} - \frac{TE 1}{2} - \frac{{T}_{T 2 P}}{4} = \frac{{T}_{T 2 P}}{4} - \frac{TE 1}{2} > 0 .$$

If τ*=T_{T2P}/8 is selected, then

$$TE 1 = \frac{{T}_{T 2 P}}{4} < \frac{{T}_{T 2 P}}{2} .$$

The extension to the recharging time of the RFPA between second RF refocusing pulse RF2 and a subsequent RF pulse RF4 which is, for example, an inversion pulse is, is thus

$$Δ tr 2 = \frac{{T}_{T 2 P}}{4} - \frac{TE 1}{2} = \frac{{T}_{T 2 P}}{4} - \frac{{T}_{T 2 P}}{8} = \frac{{T}_{T 2 P}}{8}$$

As a rule, an extension of this kind allows an adequate recharging time in order, for example, to be able to execute even further adiabatic RF pulses as the first RF pulse RF4 of an imaging sequence.

An example of this: a typical T2-preparation duration for T2-FLAIR is T_{T2P}=125 ms.

Assuming a maximum charge of a capacitor bank of a magnetic resonance device with a field strength of the main magnetic field of 0.55 T, then Q₀=1.71 Vs and the recharging rate is κ=48.3 V. This means that a capacitor bank fully discharged by an RF pulse is recharged in the time

$${t}_{c} = \frac{{Q}_{0}}{τ} = \frac{1.71 Vs}{48.3 V} = 0.0 \; 354 s = 35.4 ms .$$

If, therefore, the recharging times tr1 and tr2 are longer than t_{c}, the T2-prep module can then be implemented with a subsequent IR pulse if a single one of the RF refocusing pulses RF2 can be implemented.

A hyperbolic secant pulse with a duration T_{refoc}=10240 us can be used as the RF refocusing pulse RF2. A rectangular pulse respectively, for example with a duration T_{flip-back}=500 μs, can be used as the 90° RF excitation and 90° RF flip-back pulse. For a known T2-preparation according to FIG. 2, the following would thus apply:

$$tr 1 = \frac{{T}_{T 2 P}}{2} - {T}_{refoc} = 52.6 ms$$

$$and$$

$$tr 2 = \frac{{T}_{T 2 P}}{4} - \frac{{T}_{refoc}}{2} - \frac{{T}_{flip - b a c k}}{2} = 31.25 ms - 5.12 ms - 0.25 ms = 25.88 ms .$$

Since tr1 is longer than t_{c}, the capacitor bank should be completely charged immediately before the second RF refocusing pulse RF2 of the T2-preparation module. The recharging time tr2 is shorter than t_{c}. If a single RF refocusing pulse RF2 therefore almost fully discharges the capacitor bank, it is sometimes not possible to implement an inversion pulse RF4 which follows the T2-preparation module.

A simulation of a charge level of an RFPA for a transmitter-reference amplitude of 230V for an embodiment according to FIG. 2 results in a single hyperbolic secant pulse discharging the capacitor bank to approximately 11% of its maximum charge (Δq_{refoc}≈−0.8900). A single RF pulse can be implemented thereby. Between the first and the second RF refocusing pulse RF2, the capacitor bank is fully recharged, so the second RF refocusing pulse RF2 of the T2-preparation module can also be implemented. The capacitor bank cannot be completely recharged during the time tr2 between the second Rf-refocusing pulse RF2 and the 90° RF flip-back pulse RF3. The short recharging time tr3 between the 90° RF flip-back pulse RF3 of the T2-preparation module and the start of a subsequent RF pulse RF4 of one of the imaging sequence is also not sufficient to fully charge the capacitor bank, in particular since the smaller discharge Δq_{flip-back} due to the 90° RF flip-back pulse RF3 also has to be compensated during this time. During the playing out of an inversion pulse as the first Rf-pulse RF4 of a following imaging sequence, the charge in the simulation is negative. In reality this means that an inversion pulse RF4 of this kind cannot be implemented as specified by the sequence, or a monitoring component protection switches off. In the first case, image artifacts result, in the second case, the measurement is terminated.

A simulation of a charge level of an RFPA for a transmitter reference amplitude of 230V for an inventive embodiment according to FIG. 4, with identical T2-preparation duration T_{T2P}=125 ms and, compared to the above example according to FIG. 2, produces RF refocusing pulses RF2 shifted forwards in time by 12.5 ms (whereby the echo time TE1 of the primary spin echo TE1 is thereby shortened from 62.5 ms to 37.5 ms) that the recharging time between the two RF refocusing pulses

$$RF 2 tr 1 = \frac{{T}_{T 2 P}}{2} - {T}_{refoc} = 52.6 ms$$

is unchanged compared to the example in FIG. 2, the recharging time between the second RF refocusing pulse RF2 and the 90° RF flip-back pulse RF3 is extended by 12.5 ms, however, and is thereby tr2=25.88 ms+12.5 ms=38.38 ms. tr2 is thereby now longer than t_{c} and the capacitor bank is fully charged before the 90° RF flip-back pulse RF3. The short recharging time tr3 between the 90° RF flip-back pulse RF3 of the T2-preparation module is sufficient to compensate the comparatively small removal of charge Δq_{flip-back} of the 90° RF flip-back pulse RF3, so the capacitor bank is fully charged immediately before the first RF pulse RF4, so even an adiabatic inversion pulse can be implemented as RF4.

FIG. 5 shows a schematic graph to illustrate the time correlations of the RF pulses to be irradiated of an inventive T2-preparation module with four adiabatic RF pulses for refocusing. Compared to the graph of FIG. 1, the RF refocusing pulses RF2 are shifted forwards here. By way of example, the RF refocusing pulses RF2 can be shifted forwards by

$$Δ t = \frac{{T}_{T 2 P}}{8} - \frac{TE 1}{2}$$

compared to a known embodiment as in FIG. 1.

Compared to the graph of FIG. 1, the first RF refocusing pulse RF2 is thereby irradiated here as early as after a period τ*, where τ*<τ, so an echo time TE1 of the first spin echo E1 is again double this period τ*, TE1=2τ*. The echo time TE1 of the first spin echo E1 is thus also shorter here than the echo time (TE1=T=T_{T2P}/4) of the first spin echo E1 according to FIG. 2. The echo time TE1 of the first refocused spin echo E1 is thus shorter here than the characteristic duration T_{T2P} divided by the number (here: four) of the irradiated RF refocusing pulses RF2.

Here too, the recharging time between the RF refocusing pulses RF2, tr1, remains unchanged compared to the known embodiment according to FIG. 1. The recharging time tr2 between the most recent RF refocusing pulse RF2 of the T2-preparation module and the 90° RF flip-back pulse RF3 is again extended by

$$Δ tr 2 = \frac{{T}_{T 2 P}}{8} - \frac{TE 1}{2} > 0 ,$$

however.

If, as a whole, a readout module of an imaging sequence satisfies the condition 3 in respect of the charge of a capacitor bank, therefore −Q₀≤Δq_{readout-modul}<0, then the readout module can only be executed if the charge status of the capacitor bank immediately before the readout module is greater than or equal to |Δq_{readout-modul}|. The advantage of the inventive method also results here from the extension of the recharging time between the most recent RF refocusing pulse RF2 of the T2-preparation module and the following readout module of an imaging sequence. The condition 3 in words means that the readout module as a whole deducts more charge from the charge account of the RFPA than is recharged throughout the duration of the readout module.

For the proposed method, the echo time TE1 of the first refocused spin echo E1 is bounded below only by the duration of the RF pulses, in particular of the RF excitation pulse RF1 and of the first RF refocusing pulse RF2.

The shortening of the echo time TE1 of the first refocused spin echo E1 means a period between the irradiation of the RF excitation pulse RF1 and the first RF refocusing pulse RF2 is shorter than the characteristic duration T_{T2P} divided by double the number of irradiated RF refocusing pulses RF2. In this case, the period between the irradiation of the RF excitation pulse RF1 and the first RF refocusing pulse RF2 can, in particular, be at least or exactly half as large as the characteristic duration T_{T2P} divided by double the number of irradiated RF refocusing pulses RF2. The greater the shortening of the echo time TE1 of the first refocused spin echo E1, the more the recharging time tr2 between the most recent RF refocusing pulse RF2 and the 90° RF flip-back pulse RF3 can be increased, with a shortening by a half, as a rule, already being sufficient and it being possible to easily calculate it.

The irradiated RF refocusing pulses RF2 can be adiabatic RF pulses. A B1-sensitivity of the T2-preparation can be reduced thereby.

An even number of RF refocusing pulses RF2 can be irradiated after the RF excitation pulse RF1, in particular for adiabatic inversion pulses as the RF refocusing pulses RF2, a phase dispersion can thus be prevented or compensated.

The irradiated RF refocusing pulses RF2 can be hyperbolic secant pulses or adiabatic RF pulses of the BIREF-1 type. Hyperbolic secant pulses and adiabatic RF pulses of the BIREF-1 type are already used for various T2-preparation modules, so known T2-preparation modules can be used as the basis for novel T2-preparation modules which are described here.

The RF excitation pulse RF1 and/or the RF pulse RF3 for returning the generated transversal magnetization can be a non-selective rectangular pulse. Non-selective rectangular pulses are insensitive to inhomogeneities of the main magnetic field and cause only a slight loading of RFPAs which are used.

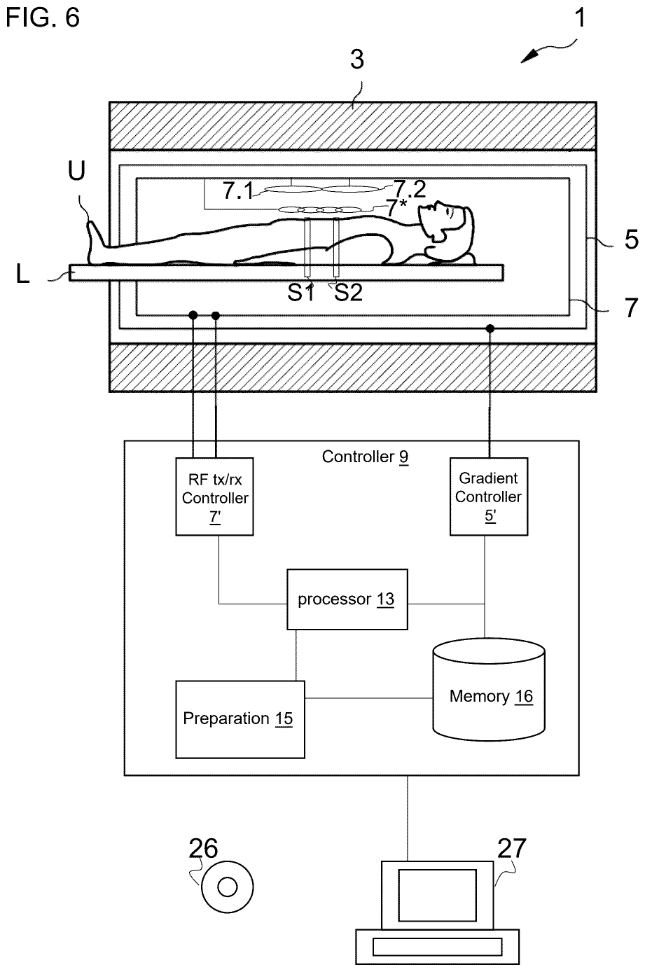

FIG. 6 schematically represents an inventive magnetic resonance system 1. This may comprise a magnet unit 3 for generating the main magnetic field, a gradient unit 5 for generating the gradient fields, a radio-frequency unit 7 for irradiation and receiving of radio-frequency signals and a control facility (controller) 9 configured to carrying out an inventive method according to the disclosure. The controller 9 may include processing circuitry configured to perform one or more functions and/or operations of the controller 9. Additionally, or alternatively, one or more components of the controller 9 may include processing circuitry that is configured to perform one or more respective functions of the component(s).

FIG. 6 only roughly schematically represents these partial units of the magnetic resonance system 1. The radio-frequency unit 7 may include a plurality of subunits and, for example, comprise a plurality of coils. In particular, the radio-frequency unit 7 can comprise a body coil which is permanently integrated in the magnetic resonance system 1, and can in turn comprise, for example, two antenna elements 7.1 and 7.2. Furthermore, the radio-frequency unit 7 can comprise one or more different local coil(s) 7*, which can be embodied either to only transmit radio-frequency signals or to only receive the triggered radio-frequency signals, or for both, and for their part can comprise a plurality of antenna elements and associated coil channels.

For examination of an examination object U, for example, a patient or also a phantom, they can be introduced on a couch L into the magnetic resonance system 1, into the measuring volume thereof. The slices S1 or S2 represent exemplary target volumes of the examination object, from which echo signals can be recorded and captured as measurement data.

The control facility (controller) 9 may be configured to control the magnetic resonance system 1 and can, in particular, control the gradient unit 5 by means of a gradient controller 5′ and the radio-frequency unit 7 by means of a radio-frequency transmit/receive controller 7′. The radio-frequency unit 7 can comprise a plurality of channels on which signals can be transmitted or received.

The radio-frequency unit 7, together with its radio-frequency transmit/receive controller 7′, is responsible for generating and irradiating (transmitting) a radio-frequency alternating field for manipulation of the spins in a region, which is to be manipulated (for example, in slices to be measured S), of the examination object U. As a rule, the center frequency of the radio-frequency alternating field, also referred to as the B1 field, is optimally adjusted such that it lies close to the resonance frequency of the spins to be manipulated. Deviations from the center frequency by the resonance frequency are referred to as off resonance. Currents controlled by means of the radio-frequency-transmit/receive controller 7′ are applied to the RF coils in the radio-frequency unit 7 in order to generate the B1 field.

Furthermore, the control facility 9 may comprise a preparation unit 15 for ascertaining inventive T2-preparation modules for preparation of a T2-weighted magnetization. As a whole, the control facility 9 may be configured to carry out an inventive method.

A computing unit (processor, processing circuitry) 13 of the control facility 9 is embodied to execute all computing operations necessary for the required measurements and determinations. Intermediate results and results required for this or ascertained in this case can be stored in a memory unit 16 of the controller 9. The represented units are not necessarily to be understood as physically separate units, rather they merely represent a subdivision into coherent units, which, however, can also be implemented, for example, in fewer or also in just a single physical unit.

Control commands can be guided to the magnetic resonance system and/or results of the control facility 9, such as image data, can be displayed, for example by a user, via an input/output facility 27 of the magnetic resonance system 1.

A method described herein can also be in the form of a computer programs which comprises commands which execute the described method on a control facility 9. A computer-readable storage medium can also be present which comprises commands which, when executed by a control facility 9 of a magnetic resonance system 1, prompt it to execute the described method.

To enable those skilled in the art to better understand the solution of the present disclosure, the technical solution in the embodiments of the present disclosure is described clearly and completely below in conjunction with the drawings in the embodiments of the present disclosure. Obviously, the embodiments described are only some, not all, of the embodiments of the present disclosure. All other embodiments obtained by those skilled in the art on the basis of the embodiments in the present disclosure without any creative effort should fall within the scope of protection of the present disclosure.

It should be noted that the terms “first”, “second”, etc. in the description, claims and abovementioned drawings of the present disclosure are used to distinguish between similar objects, but not necessarily used to describe a specific order or sequence. It should be understood that data used in this way can be interchanged as appropriate so that the embodiments of the present disclosure described here can be implemented in an order other than those shown or described here. In addition, the terms “comprise” and “have” and any variants thereof are intended to cover non-exclusive inclusion. For example, a process, method, system, product or equipment comprising a series of steps or modules or units is not necessarily limited to those steps or modules or units which are clearly listed, but may comprise other steps or modules or units which are not clearly listed or are intrinsic to such processes, methods, products or equipment.

References in the specification to “one embodiment,” “an embodiment,” “an exemplary embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The exemplary embodiments described herein are provided for illustrative purposes, and are not limiting. Other exemplary embodiments are possible, and modifications may be made to the exemplary embodiments. Therefore, the specification is not meant to limit the disclosure. Rather, the scope of the disclosure is defined only in accordance with the following claims and their equivalents.

Embodiments may be implemented in hardware (e.g., circuits), firmware, software, or any combination thereof. Embodiments may also be implemented as instructions stored on a machine-readable medium, which may be read and executed by one or more processors. A machine-readable medium may include any mechanism for storing or transmitting information in a form readable by a machine (e.g., a computer). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Further, firmware, software, routines, instructions may be described herein as performing certain actions. However, it should be appreciated that such descriptions are merely for convenience and that such actions in fact results from computing devices, processors, controllers, or other devices executing the firmware, software, routines, instructions, etc. Further, any of the implementation variations may be carried out by a general-purpose computer.

The various components described herein may be referred to as “modules,” “units,” or “devices.” Such components may be implemented via any suitable combination of hardware and/or software components as applicable and/or known to achieve their intended respective functionality. This may include mechanical and/or electrical components, processors, processing circuitry, or other suitable hardware components, in addition to or instead of those discussed herein. Such components may be configured to operate independently, or configured to execute instructions or computer programs that are stored on a suitable computer-readable medium. Regardless of the particular implementation, such modules, units, or devices, as applicable and relevant, may alternatively be referred to herein as “circuitry,” “controllers,” “processors,” or “processing circuitry,” or alternatively as noted herein.

For the purposes of this discussion, the term “processing circuitry” shall be understood to be circuit(s) or processor(s), or a combination thereof. A circuit includes an analog circuit, a digital circuit, data processing circuit, other structural electronic hardware, or a combination thereof. A processor includes a microprocessor, a digital signal processor (DSP), central processor (CPU), application-specific instruction set processor (ASIP), graphics and/or image processor, multi-core processor, or other hardware processor. The processor may be “hard-coded” with instructions to perform corresponding function(s) according to aspects described herein. Alternatively, the processor may access an internal and/or external memory to retrieve instructions stored in the memory, which when executed by the processor, perform the corresponding function(s) associated with the processor, and/or one or more functions and/or operations related to the operation of a component having the processor included therein.

In one or more of the exemplary embodiments described herein, the memory is any well-known volatile and/or non-volatile memory, including, for example, read-only memory (ROM), random access memory (RAM), flash memory, a magnetic storage media, an optical disc, erasable programmable read only memory (EPROM), and programmable read only memory (PROM). The memory can be non-removable, removable, or a combination of both.

1. A method for preparation of a T2-weighted magnetization in an examination object for magnetic resonance imaging, comprising:
irradiating a radio frequency (RF) excitation pulse configured to generate a transversal magnetization in the examination object;
after irradiation of the RF excitation pulse, irradiating at least two RF refocusing pulses configured to refocus the generated magnetization, a spin echo being formed after each RF refocusing pulse of the at least two RF refocusing pulses, and
after a most recent irradiated RF refocusing pulse of the at least two RF refocusing pulses, and after a first duration, based on a prepared T2-weighting, after the irradiation of the RF excitation pulse, irradiating an RF pulse configured to return the generated transversal magnetization into the longitudinal,
wherein the at least two RF refocusing pulses are irradiated such that an echo time of a first refocused spin echo is shorter than a second duration that is based on the first duration and a number of RF refocusing pulses, of the at least two RF refocusing pulses, having been irradiated.

2. The method as claimed in claim 1, wherein the at least two irradiated RF refocusing pulses are adiabatic RF pulses.

3. The method as claimed in claim 2, wherein the number of RF refocusing pulses is an even number of RF refocusing pulses.

4. The method as claimed in claim 1, wherein the at least two irradiated RF refocusing pulses are hyperbolic secant pulses.

5. The method as claimed in claim 1, wherein the at least two irradiated RF refocusing pulses are adiabatic RF pulses of the BIREF-1 type.

6. The method as claimed in claim 1, wherein the RF excitation pulse and/or the RF pulse is a non-selective rectangular pulse.

7. The method as claimed in claim 1, wherein a period between the irradiation of the RF excitation pulse and a first of the at least two RF refocusing pulses is shorter than the second duration.

8. The method as claimed in claim 1, wherein a period between the irradiation of the RF excitation pulse and a first of the at least two RF refocusing pulses is at least half as short as the second duration.

9. The method as claimed in claim 1, wherein immediately after the RF pulse, a first RF pulse of an imaging sequence is irradiated so as to perform the imaging sequence, to generate image data of the examination object, immediately after preparation of the T2-weighted magnetization for capturing measurement data.

10. The method as claimed in claim 1, further comprising irradiating, based on the RF pulse, a first RF pulse of an imaging sequence to generate image data of the examination object.

11. The method as claimed in claim 10, wherein the first RF pulse of an imaging sequence is irradiated immediately after preparation of the T2-weighted magnetization.

12. The method as claimed in claim 1, wherein the second duration is a quotient of the first duration and the number of RF refocusing pulses.

13. A magnetic resonance system comprising: a scanner; and a controller configured to control the scanner and to perform the method of claim 1.

14. The magnetic resonance system as claimed in claim 13, wherein the scanner comprises a magnet unit, a gradient unit, and an RF unit.

15. An apparatus comprising:
one or more processors; and
memory storing instructions that, when executed by the one or more processors, cause the apparatus to perform the method of claim 1.

16. One or more non-transitory media storing instructions that, when executed by one or more processors, cause the one or more processors to perform the method of claim 1.
